(19) United States
(12) Patent Application Publication
(10) Pub. No.: US 2023/0190851 A1
(43) Pub. Date: Jun. 22, 2023

(54) PLANT COMPOUNDS FOR TREATING ALZHEIMER'S DISEASE

(71) Applicants: Frederikus Gerardus Josephus Maria VAN DE WIEL, Waalwijk (NL); RRW B.V., Waalwijk (NL)

(72) Inventors: Frederikus Gerardus Josephus Maria VAN DE WIEL, Waalwijk (NL); Marjolein WILDWATER, Heerhugowaard (NL)

(21) Appl. No.: 17/926,161

(22) PCT Filed: May 18, 2021
(86) PCT No.: PCT/EP2021/063117

§ 371 Date: Nov. 18, 2022

(30) Foreign Application Priority Data
May 18, 2020 (DE) 10 2020 002 947.7

(51) Int. Cl. A61K 36/54; A61K 36/185; A61K 36/9066; A61P 25/28

(52) U.S. Cl. CPC A61K 36/54; A61K 36/185; A61K 36/9066; A61P 25/28

(57) ABSTRACT

The present invention relates to the treatment or prevention of amyloidosis using a combination of herbal active agents. In particular, a curcuminoid and/or a cannabinoid are used together with cinnamon to treat or prevent Alzheimer's disease.

### CROSS-REFERENCE TO RELATED APPLICATIONS

This application is a 35 U.S.C. § 371 national stage filing of International Application No. PCT/EP2021/063117 filed on May 18, 2021, which claims the benefit of German Patent Application No. 10 2020 002 947.7, filed on May 18, 2020. The entire contents of each of these applications are incorporated herein by reference in their entirety.

### FIELD OF THE INVENTION

The present invention relates to the technical field of treatment of amyloidoses such as Alzheimer's disease. In particular, the invention relates to therapies with cannabinoids and/or curcuminoids in combination with cinnamon.

### TECHNICAL BACKGROUND

One of the most common forms of neurodegenerative disease is Alzheimer's disease, which is also known synonymously as Morbus Alzheimer. Alzheimer's disease occurs particularly in older people beyond the age of 65, although the corresponding precursor forms or preclinical or clinical precursors may occur much earlier.

The prevalence of neurodegenerative diseases, and Alzheimer's disease in particular, is strongly increasing and its prevalence is steadily increasing worldwide, which will place severe strains on public health systems in the future. Alzheimer's disease is the most widespread form of dementia. Alzheimer's disease is associated with progressive deterioration of cognitive performance over time, usually accompanied by a decline in daily activities, behavioral abnormalities, and neuropsychological symptoms.

Characteristic of Alzheimer's disease are the plaques that form in the affected person's brain, which essentially have misfolded amyloid-β peptides, and the neurofibrils that accumulate in the neurons. The intracellularly located neurofibril bundles are substantially composed of the tau protein, which aggregates into fibrils when it is excessively phosphorylated. The amyloid-β peptides, which are also synonymously referred to as Aβ proteins, are formed from the so-called amyloid precursor protein (APP). APP is a type I transmembrane protein that is cleaved by certain proteinases. Cleavage of APP results in the release of Aβ-peptides from the precursor protein. Aβ-peptides are generally 37 to 42 amino acids in length and are associated with the pathological formation of Alzheimer's disease.

Deposits of proteins such as the amyloid protein or the tau protein also play a crucial role in other diseases. The protein deposits (amyloids) cause damage to cells and tissues, with the clinical picture being defined according to the location and type of deposition.

The objective of the present invention is to provide an improved prevention or treatment of these amyloidoses, in particular Alzheimer's disease.

### SUMMARY OF THE INVENTION

The invention is based on the finding that the therapeutic effect of various active agents in the treatment of amyloidoses such as Alzheimer's disease can be enhanced by combining them with cinnamon. Synergistic effects of these combination therapies have been demonstrated in an animal model.

Based on this finding, in a first aspect, the present invention provides the use of cinnamon for the manufacture of a medicament for the treatment or prevention of amyloidosis, wherein the cinnamon is administered in combination with an active agent that is therapeutically effective in the treatment or prevention of amyloidosis.

In a second aspect, the present invention relates to the use of a mixture of cinnamon and a cannabinoid and/or a curcuminoid for the manufacture of a medicament for the treatment or prevention of amyloidosis.

In a third aspect, the present invention relates to the use of a cannabinoid for the manufacture of a medicament for the treatment or prevention of amyloidosis, wherein the cannabinoid is administered in combination with cinnamon and optionally a curcuminoid.

In a fourth aspect, the present invention relates to the use of a curcuminoid for the manufacture of a medicament for the treatment or prevention of amyloidosis, wherein the curcuminoid is administered in combination with cinnamon and optionally a cannabinoid.

In a fifth aspect, the present invention relates to the use of cinnamon for the manufacture of a medicament for the treatment or prevention of amyloidosis, wherein the cinnamon is administered in combination with a curcuminoid and/or a cannabinoid.

Other purposes, features, advantages and aspects of the present application will be apparent to those skilled in the art from the following description and the appended claims. Various changes and modifications in the spirit and scope of the disclosed invention will be apparent to those skilled in the art from the following disclosure.

### DETAILED DESCRIPTION OF THE INVENTION

Cannabinoid compounds such as cannabidiol (CBD) and curcuminoid compounds such as curcumin showed beneficial effects in the treatment and prevention of amyloidosis in an animal model. With the additional addition of cinnamon, these effects were surprisingly significantly increased. The combination of a cannabinoid or a curcuminoid with cinnamon can thus be used in the treatment or prevention of amyloidoses such as Alzheimer's disease, with a significantly improved therapeutic effect obtained by the use of cinnamon.

With this in mind, in a first aspect, the present invention provides the use of cinnamon for the manufacture of a medicament for the treatment or prevention of amyloidosis, wherein the cinnamon is administered in combination with an active agent that is therapeutically effective in the treatment or prevention of amyloidosis.

The active agent that is therapeutically effective in the treatment or prevention of amyloidosis may be any therapeutically effective active agent, particularly an active agent that can be used to treat and/or prevent Alzheimer's disease. In particular embodiments, the active agent is a plant-derived active agent or an active agent obtainable from plants. In particular, the active agent can also be a mixture of different substances. In certain embodiments, the active agent is a cannabinoid, a curcuminoid, or a mixture thereof.

In a second aspect, the present invention relates to the use of a mixture of cinnamon and a cannabinoid and/or a curcuminoid for the manufacture of a medicament for the treatment or prevention of amyloidosis.

In particular, the present invention relates to the use of a mixture of a cannabinoid and cinnamon for the manufacture of a medicament for the treatment or prevention of amyloidosis.

Further, the present invention relates to the use of a mixture of a curcuminoid and cinnamon for the manufacture of a medicament for the treatment or prevention of amyloidosis.

In addition, the present invention relates to the use of a mixture of a cannabinoid, a curcuminoid and cinnamon for the manufacture of a medicament for the treatment or prevention of amyloidosis.

In a third aspect, the present invention relates to the use of a cannabinoid for the manufacture of a medicament for the treatment or prevention of amyloidosis, wherein the cannabinoid is administered in combination with cinnamon and optionally a curcuminoid.

In a fourth aspect, the present invention relates to the use of a curcuminoid for the manufacture of a medicament for the treatment or prevention of amyloidosis, wherein the curcuminoid is administered in combination with cinnamon and optionally a cannabinoid.

In a fifth aspect, the present invention relates to the use of cinnamon for the manufacture of a medicament for the treatment or prevention of amyloidosis, wherein the cinnamon is administered in combination with a curcuminoid and/or a cannabinoid.

In these aspects of the invention, the cinnamon, cannabinoid, and/or curcuminoid may be present together in a mixture and administered to the patient. Alternatively, the cinnamon, cannabinoid and curcuminoid may each be present in a separate composition and administered to the patient. Moreover, in the combination of cinnamon, cannabinoid, and curcuminoid, two of the substances may be present together in a mixture, and the third substance may be present either alone in a composition or together with one of the other two substances in a mixture.

An administration of the individual substances in combination with one another also comprises embodiments in which the substances are administered to the patient separately from one another in separate compositions. Administration may be simultaneous or sequential. Consecutive administration is particularly performed on the same day, preferably within a period of 4 hours, 2 hours, or more preferably one hour.

In particular, a therapeutic composition is a composition suitable for administration to a human or animal. A therapeutic composition consists in particular of pharmaceutically acceptable substances. Preferably, a pharmaceutical composition contains an active agent, in particular a cannabinoid and/or a curcuminoid, and a carrier, a solvent, and/or further additives such as buffers and preservatives. In particular embodiments, the therapeutic composition contains only water in addition to the specified substances cannabinoid, curcuminoid, and/or cinnamon.

For purposes of the present invention, curcuminoids in particular comprise curcumin, demethoxycurcumin and bisdemethoxycurcumin, and mixtures thereof. In a particular embodiment, the curcuminoid is curcumin or a mixture containing curcumin. In a particular embodiment, the curcuminoid is a mixture containing curcumin, optionally demethoxycurcumin, and optionally bisdemethoxycurcumin. For example, the curcuminoid may be a mixture containing at least 60%, in particular at least 70% curcumin, at most 30%, in particular at most 20% demethoxycurcumin, and at most 10%, in particular at most 5% bisdemethoxycurcumin. Curcumin is also known as diferuloylmethane or (E,E)-1,7-bis-(4-hydroxy-3-methoxyphenyl)-1,6-heptadiene-3,5,-dione. The curcuminoid, in particular curcumin, can be obtained from a natural source such as the rhizome of the plant turmeric (Curcuma longa) or synthetically.

For purposes of the present invention, cannabinoids in particular comprise cannabidiol (CBD), delta-9-tetrahydrocannabinol (THC), cannabichromene, cannabielsoin, delta-8-tetrahydrocannabinol, iso-tetrahydrocannabinol, cannabicyclol, cannabicitran, and acids of these compounds such as cannabidiolic acid (CBDA) and delta-9-tetrahydrocannabinolic acid (THCA), and mixtures thereof. In a particular embodiment, the cannabinoid is CBD or a mixture containing CBD. In a further embodiment, the cannabinoid is THC or a mixture containing THC. In a further embodiment, the cannabinoid is THCA or a mixture containing THCA. The cannabinoid, particularly CBD, THC, and/or THCA, can be obtained from a natural source such as the hemp plant or synthetically.

For the purposes of the present invention, cinnamon is in particularly a mixture of substances obtainable from the bark of a cinnamon tree of the genus Cinnamomum. In particular, this mixture of substances may contain one or more of the substances selected from the group consisting of cinnamaldehyde, trans-cinnamaldehyde, cinnamic acid and essential oils, including trans-cinnamaldehyde, cinnamyl acetate, eugenol, L-borneol, caryophyllene oxide, B-caryophyllene, L-bornyl acetate, E-nerolidol, α-cubebene, α-terpineol, terpinolene and α-thujene. Cinnamon can be obtained from natural sources or produced synthetically. In a particular embodiment, cinnamon contains cinnamaldehyde. In one particular embodiment, cinnamon is cinnamaldehyde. Cinnamon, in particular cinnamaldehyde, can be obtained from a natural source such as the bark of a cinnamon tree or synthetically.

In certain embodiments, the cannabinoid, curcuminoid, and cinnamon, or the mixture containing the cannabinoid, curcuminoid, and/or cinnamon, are used in the form of a therapeutic composition. In certain embodiments, the cinnamon and the cannabinoid and/or the curcuminoid are present together in a therapeutic composition.

The administration to the patient can be done by any suitable route of administration. In particular embodiments, the substances or mixtures of substances, in particular the therapeutic compositions, are administered orally.

In particular, treatment or prevention is achieved by regular administration over an extended period of time, preferably continuously. In one embodiment, the substances are administered in a therapeutically effective amount.

Amyloidosis is a disease with accumulation of proteins. The enriched proteins form insoluble depositions (amyloid) such as fibrils. In particular embodiments, amyloidosis is a tauopathy. In tauopathies, tau protein is deposited in nerve and glial cells. In particular, the group of tauopathies comprises Alzheimer's disease, chronic traumatic encephalopathy (CTE), Parkinson's disease, corticobasal degeneration, and frontotemporal dementia. In a preferred embodiment, the amyloidosis is Alzheimer's disease. In Alzheimer's disease, plaques composed of misfolded beta-amyloid (Aβ) peptides form in the affected person's brain, and faulty tau proteins attach to axons from within the cell body.

Treatment is given to a patient in need thereof. These are in particular patients suffering from amyloidosis such as Alzheimer's disease. The treatment can be given at the onset of the disease as well as in advanced disease. Prevention can be done for any individual. In particular, prevention is applied to individuals of older age, such as from the age of 60, 65, 70 or 75. However, individuals of lesser ages may also benefit from prevention, such as individuals at least 20 years of age, at least 30 years of age, at least 40 years of age, or at least 50 years of age. In individuals with incipient or undiagnosed amyloidosis, prevention and treatment can be intertwined. The patients and individuals are in particular humans.

In certain embodiments, the cinnamon, curcuminoid, and/or cannabinoid are administered in one or more pharmaceutical compositions containing substantially only water in addition to these substances. In specific embodiments, the pharmaceutical compositions contain only water in addition to these substances. In certain embodiments, the pharmaceutical compositions contain these substances in high concentrations. For example, the concentration of curcuminoid, in particular curcumin, is at least 1 g/I, e.g., at least 2 g/I, at least 3 g/I, at least 5 g/I, at least 10 g/I, at least 15 g/I, or approximately 20 g/I. For example, the concentration of the cinnamon may be at least 1 g/I, in particular at least 5 g/I, at least 10 g/I, at least 20 g/I, at least 50 g/I or at least 100 g/I. For example, the concentration of the cannabinoid, in particular of CBD, may be at least 0.5 mg/I, preferably at least 1 mg/I, at least 10 mg/I, at least 100 mg/I, or at least 1 g/I.

The term “containing” or related expressions, where used herein, shall mean, in addition to the ordinary meaning thereof, “consisting essentially of” and “consisting of”. Thus, the term “containing” also refers to embodiments in which the subject matter “containing” certain listed elements does not contain further elements, as well as embodiments in which the subject matter “containing” certain listed elements additionally contains further elements. The term “consisting essentially of” relates in particular to embodiments in which the subject matter contains 10% or less, in particular 5% or less, additional elements in addition to the specifically listed elements of which the subject matter consists essentially.

### FIGURES

FIG. 1 shows the results of an Alzheimer's disease paralysis assay. A C. elegans nematode strain with inducible amyloid-β accumulation (CL4176) was exposed to curcumin (compound A), cinnamon (compound D), and a combination of curcumin and cinnamon. The percentage of nematodes that have not yet become immobile as a result of Aβ accumulation is given as a function of time from induction of Aβ accumulation. Untreated nematodes or nematodes treated only with the solvent (water) served as controls.

FIG. 2 shows the results of an Alzheimer's disease paralysis assay. A C. elegans nematode strain with inducible amyloid-β accumulation (CL4176) was exposed to CBD (compound B), cinnamon (compound D), and a combination of CBD and cinnamon. The percentage of nematodes that have not yet become immobile as a result of Aβ accumulation is given as a function of time from induction of Aβ accumulation. Untreated nematodes or nematodes treated only with the solvent (water) served as controls.

FIG. 3 shows the results of an Alzheimer's disease paralysis assay. A C. elegans nematode strain with inducible amyloid-β accumulation (CL4176) was exposed to THCA, cinnamon, and a combination of THCA and cinnamon. The percentage of nematodes that have not yet become immobile as a result of Aβ accumulation is given as a function of time from induction of Aβ accumulation. Untreated nematodes or nematodes treated only with the solvent (water) served as controls.

FIG. 4 shows the results of an Alzheimer's disease touch reflex assay. A C. elegans nematode strain with amyloid-β accumulation (CL2006) was exposed to THCA, cinnamon, and a combination of THCA and cinnamon for 24 hours. The percentage of nematodes that still show a response to touch and have not yet become immobilized as a result of Aβ accumulation is indicated. Untreated nematodes or nematodes treated only with the solvent (water) served as controls.

### EXAMPLES

### Example 1

### Combination of Curcumin and Cinnamon

The effects of active agents and their combinations have been studied in C. elegans nematode strains as animal models of amyloidoses such as Alzheimer's disease. These strains accumulate amyloid-β peptides that form plaques in the nematodes and lead to paralysis of the animals. In certain strains, such as strain CL4176, Aβ accumulation occurs only in response to heat shock. Thus, the Alzheimer's disease pattern is inducible in these strains. The number of paralyzed nematodes after a certain period of time following induction of Aβ accumulation indicates the extent of Alzheimer's disease. Thus, the effect of certain substances to which the nematodes are exposed on the number of paralyzed animals is a measure of the anti-Alzheimer effect of these substances.

Nematodes of strain CL4176 were synchronized at the young larval stage and allowed to grow to the L4 stage on normal growth medium for 72 h at 16° C. Then the nematodes were exposed to the test substance and the temperature was increased to 25° C. This heat shock induced Aβ accumulation, simulating the onset of Alzheimer's disease. Approximately every 24 h, the number of animals that were still mobile and those that were paralyzed as a result of Aβ accumulation was determined.

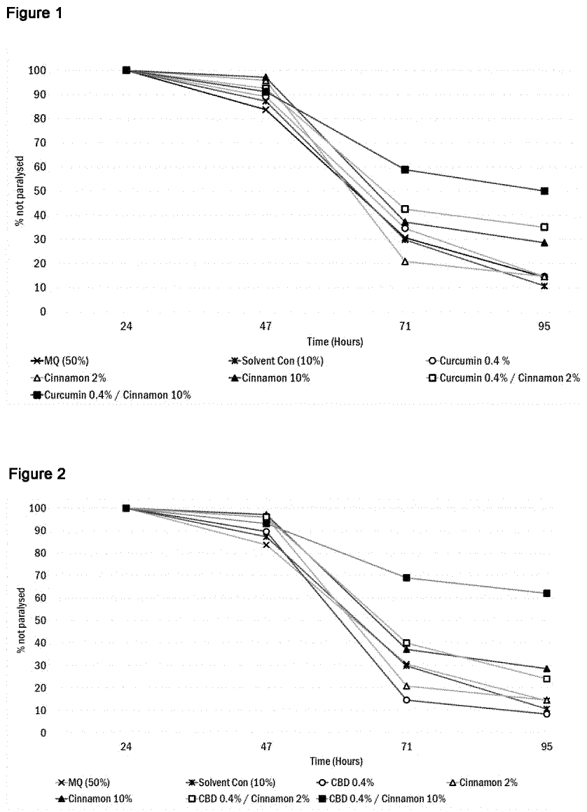

A sharp decrease in mobile “healthy” animals was observed 48 h after induction of Aβ accumulation. The untreated nematodes and the solvent (water) treated control showed consistent levels. Treatment of animals with curcumin or cinnamon alone showed little change in the number of mobile animals. However, when curcumin and cinnamon were combined, a sharp decrease in paralytic animals was observed after 71 h, which persisted for 95 h after induction of Aβ accumulation (see FIG. 1). A higher cinnamon concentration also resulted in a stronger effect. Compared with the effect of the individual active agents, the combination of curcumin and cinnamon was clearly synergistic. Thus, the combination of curcumin and cinnamon protects against the effects of Aβ accumulation and helps prevent and treat amyloidosis such as Alzheimer's disease.

### Example 2

### Combination of CBD and Cinnamon

In the assay described in Example 1, the combination of cannabidiol (CBD) and cinnamon was also examined. Once again, the individual active agents alone showed only little effect in the concentrations tested. However, the combination of CBD and cinnamon resulted in a strong synergistic increase in the number of mobile animals particularly at the higher cinnamon concentration (see FIG. 2). The combination of CBD and cinnamon thus has a therapeutic effect against amyloidoses such as Alzheimer's disease.

### Example 3

### Combination of THCA and Cinnamon

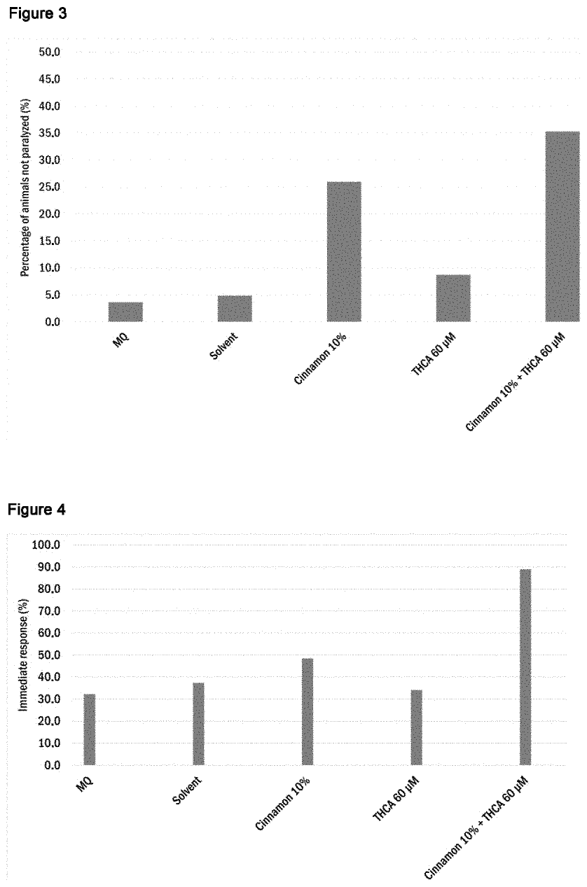

The therapeutic effect of the combination of delta-9-tetrahydrocannabinolic acid (THCA) and cinnamon was examined in the C. elegans Alzheimer's disease model. In the paralysis assay described in Example 1, the combination of THCA and cinnamon showed a synergistic effect (see FIG. 3).

In addition, the effect of this combination on the additional C. elegans strain CL2006 was examined in another assay. In this strain, nematodes accumulate Alzheimer's plaques in muscle cells during development. This leads to defects in the touch reflexes, in particular during backward movement after a light touch to the head.

For the touch reflex assay, nematodes were allowed to grow to the L4 larval stage and then exposed to the substance or mixture of substances to be tested. Response to touch was determined in a large number of animals 24 h after addition of the active agents. The percentage of animals that showed a direct response to the touch stimulus was used as a measure of the effect of Aβ accumulation in the animals. The more animals that still show a response after a certain period of time, the lower the extent of impairment due to progressive Aβ accumulation.

The data show that the combination of THCA and cinnamon also has a synergistic effect in this assay with C. elegans strain CL2006 (see FIG. 4). The combination of THCA and cinnamon thus has a therapeutic effect against amyloidoses such as Alzheimer's disease.

1. Use of cinnamon for the manufacture of a medicament for treating or preventing amyloidosis, wherein the cinnamon is administered in combination with an active agent that is therapeutically effective in treating or preventing amyloidosis.

2. Use of a mixture of a cannabinoid and cinnamon for the manufacture of a medicament for the treatment or prevention of amyloidosis.

3. Use of a cannabinoid for the manufacture of a medicament for the treatment or prevention of amyloidosis, wherein the cannabinoid is administered in combination with cinnamon and optionally a curcuminoid.

4. Use of a mixture of a curcuminoid and cinnamon for the manufacture of a medicament for the treatment or prevention of amyloidosis.

5. Use of a curcuminoid for the manufacture of a medicament for the treatment or prevention of amyloidosis, wherein the curcuminoid is administered in combination with cinnamon and optionally a cannabinoid.

6. Use of cinnamon for the manufacture of a medicament for the treatment or prevention of amyloidosis, wherein the cinnamon is administered in combination with a curcuminoid and/or a cannabinoid.

7. Use of a mixture of a cannabinoid, a curcuminoid and cinnamon for the manufacture of a medicament for the treatment or prevention of amyloidosis.

8. Use in accordance with any of claims 2, 3 and 5 to 7, wherein the cannabinoid is a mixture containing cannabidiol and/or THCA.

9. Use in accordance with any of claims 2, 3 and 5 to 7, wherein the cannabinoid is cannabidiol.

10. Use in accordance with any of claims 2, 3 and 5 to 7, wherein the cannabinoid is THCA.

11. Use in accordance with any of claims 3 to 10, wherein the curcuminoid is a mixture containing curcumin.

12. Use in accordance with any of claims 3 to 10, wherein the curcuminoid is curcumin.

13. Use in accordance with any of claims 1 to 12, wherein the cinnamon is a mixture containing cinnamaldehyde.

14. Use in accordance with any of claims 1 to 12, wherein the cinnamon is cinnamaldehyde.

15. Use in accordance with any of claims 1 to 14, wherein the cinnamon, the curcuminoid and/or the cannabinoid is contained in a pharmaceutical composition.

16. Use in accordance with claim 15, wherein the pharmaceutical composition contains only water in addition to the cinnamon, the curcuminoid, and the cannabinoid.

17. Use in accordance with any of claims 1 to 16, wherein the amyloidosis is Alzheimer's disease.
